(19) United States
(12) Patent Application Publication
(10) Pub. No.: US 2010/0261913 A1
(43) Pub. Date: Oct. 14, 2010

(54) METHOD FOR PRODUCING PRECURSORS FOR L-3,4-DIHYDROXY-6- [18F] FLUOROPHENYL ALAINE AND 2- [18F] FLUORO-L-TYROSINE AND THE ALPHA-METHYLATED DERIVATIVES THEREOF, PRECURSOR, AND METHOD FOR PRODUCING L-3, 4DIHYDROXY-6- [18F] FLUOROPHENYLALANINE AND 2- [18F] FLUORO-L-TYROSINE AND THE ALPHA-METHYLATED DERIVATIVES FROM THE PRECURSOR

(76) Inventors: Franziska Wagner, Hergolding (DE); Johannes Ermert, Koeln (DE); Heinrich Hubert Coenen, Grevenbroich (DE)

Correspondence Address: JORDAN AND HAMBURG LLP, 122 EAST 42ND STREET, SUITE 4000, NEW YORK, NY, 10168, US

(21) Appl. No.: 12/734,936

(22) PCT Filed: Nov. 21, 2008
(86) PCT No.: PCT/DE2008/001930

§ 371 Date: Jun. 25, 2010

(30) Foreign Application Priority Data
Dec. 7, 2007 (DE) 10 2007 059 313.0

(51) Int. Cl. C07D 233/32; C07C 227/40

(52) U.S. Cl. 548/322.5; 562/445; 562/446

(57) ABSTRACT

Disclosed is a method for producing precursors for L-3,4-dihydroxy-6-[¹⁸F]fluorophenylalanine and 2-[¹⁸F]fluoro-L-tyrosine and the α-methylated derivatives thereof, the precursor, and to a method for producing L-3,4-dihydroxy-6-[¹⁸F]fluorophenylalanine and 2-[¹⁸F]fluoro-L-tyrosine and the α-methylated derivatives thereof from the precursor. A compound of formula (3) is provided which enables automated synthesis of L-3,4-dihydroxy-6-[¹⁸F]fluorophenylalanine and 2-[¹⁸F]fluoro-L-tyrosine. The enantiomeric purity of the product is ≧98%.

The invention relates to a method for producing precursors for L-3,4-dihydroxy-6-[¹⁸F]fluorophenylalanine and 2-[¹⁸F]fluoro-L-tyrosine and the α-methylated derivatives thereof, to the precursor, and to a method for producing L-3,4-dihydroxy-6-[¹⁸F]fluorophenylalanine and 2-[¹⁸F]-fluoro-L-tyrosine and the α-methylated derivatives thereof from the particular precursor.

The potential of aromatic amino acids, and in particular of L-3,4-dihydroxy-6-[¹⁸F]-fluorophenylalanine (=[¹⁸F]FDOPA), in PET has been known for twenty years and has also been clinically established internationally, in particular for the examination of patients with Parkinson's disease and, over the last few years, for tumor diagnostics (amino acid transport). Overall, the tremendous interest in simple methods for the synthesis of [¹⁸F]FDOPA by conversion using easy-to-produce [¹⁸F]fluoride has been unchanged for neurological and oncological applications.

A publication from the year 2001 by T. Tierling, K. Hamacher, and H. H. Coenen entitled “A new nucleophilic asymmetric synthesis of 6-[¹⁸F]Fluoro-DOPA”, which was published in J. Label. Compds. Radiopharm. 44, Suppl. 1 is known and describes a method for producing an [¹⁸F]FDOPA precursor and the conversion thereof into L-6-[¹⁸F]fluoro-DOPA. According to this method, a precursor is first converted into precursors with a K¹⁸F cryptand by nucleophilic substitution and then into [¹⁸F]DFOPA. The product obtained has an enantiomeric purity of 85%.

Present multistage nucleophilic synthesis paths require a great deal of time and high equipment-related costs. In the previously known direct nucleophilic labeling methods, enantiomeric purities of only 85% were achieved, as in the synthesis according to Tierling, Hamacher, and Coenen. Electrophilic reaction approaches only allow radiosyntheses using activity levels that are approximately ten times lower, but involve relatively high costs for the production of the radionuclide, because the production rates of the elemental [¹⁸F]F₂ required for the electrophilic radiofluorination are considerably lower at equal irradiation costs.

The multistage nucleophilic methods are difficult to automate and prone to problems.

The methods for producing [¹⁸F]FDOPA and [¹⁸F]FTyr precursors known from the prior art result in relatively low radiochemical yields and are associated with high cost.

It is therefore the object of the invention to provide a method for producing [¹⁸F]FDOPA, [¹⁸F]FTyr and the α-methylated derivatives thereof, the precursors thereof, and a method for producing the precursors thereof, which result in greater radiochemical yields and higher enantiomeric purity. In addition, the method should be suited for automated synthesis.

The object is achieved according to the invention by the characteristics of the independent claims.

The methods and precursors according to the invention make it possible to produce [¹⁸F]FDOPA, [¹⁸F]FTyr, and the α-methylated derivatives thereof in an enantiopure manner in only three radioactive steps. The synthesis can be carried out in an automated manner and produces enantiomeric purities of ≧98%.

The figures show chemical equations for producing the precursors according to the invention and for producing the target compounds [¹⁸F]FDOPA and [¹⁸F]FTyr and the α-methylated derivatives thereof.

Shown are:

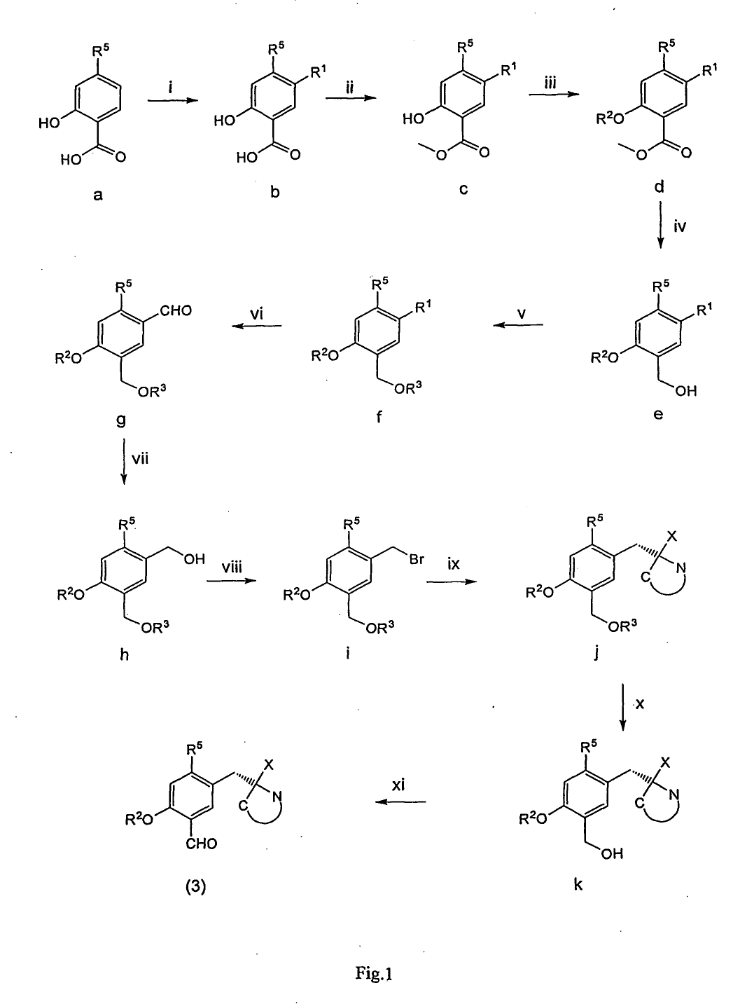

FIG. 1: A general chemical equation for producing the precursor.

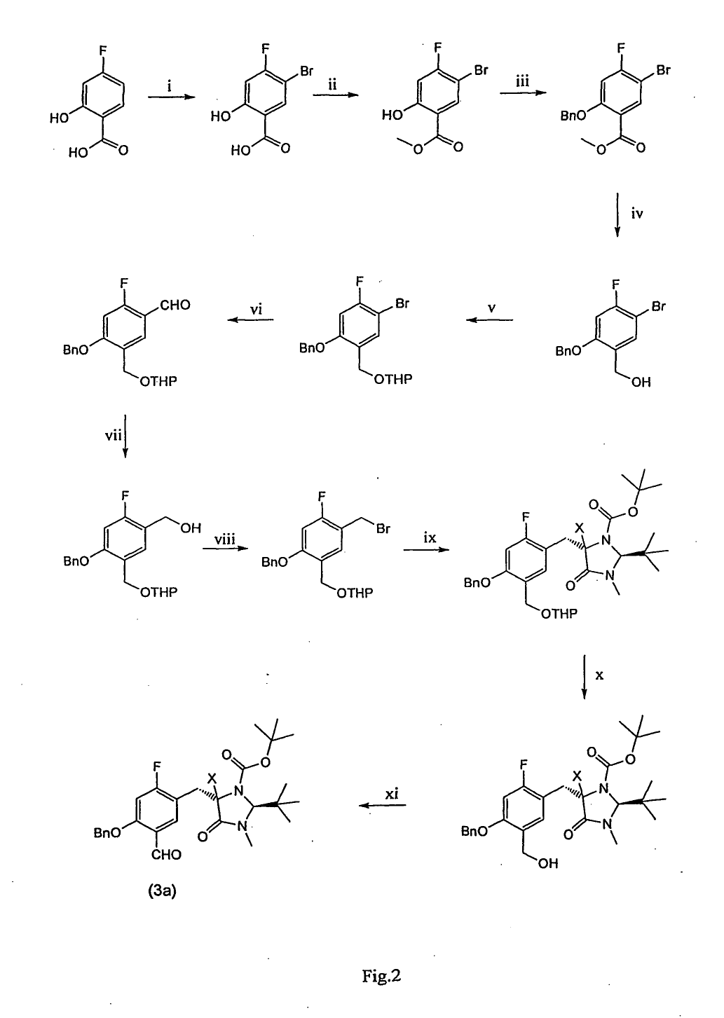

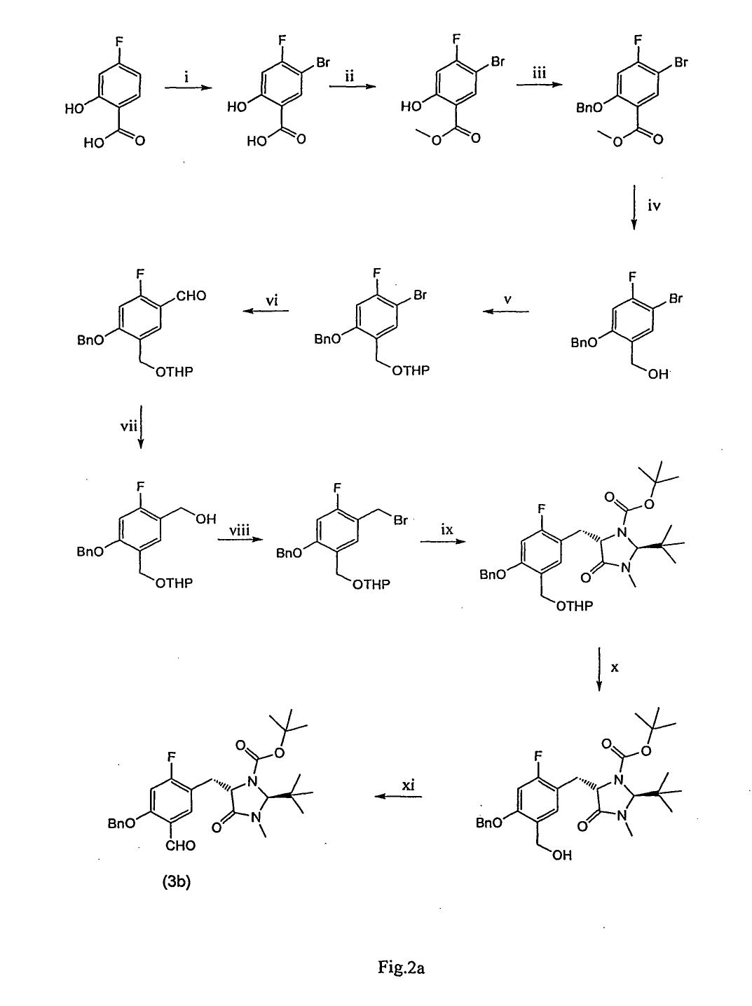

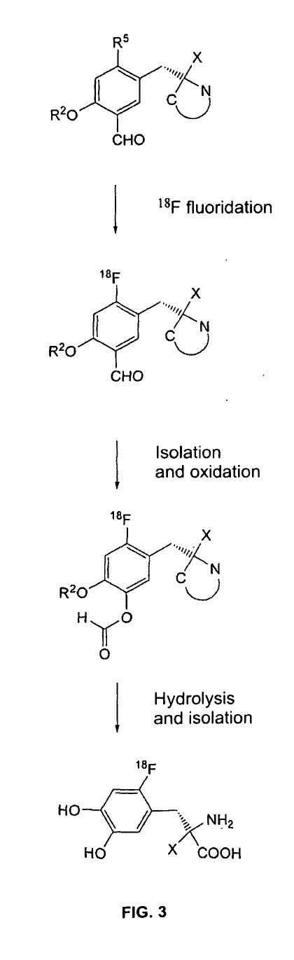

FIG. 2: A chemical equation for the synthesis of the precursor, comprising individual reaction steps.

FIG. 2a: A special exemplary embodiment for the synthesis of the precursor.

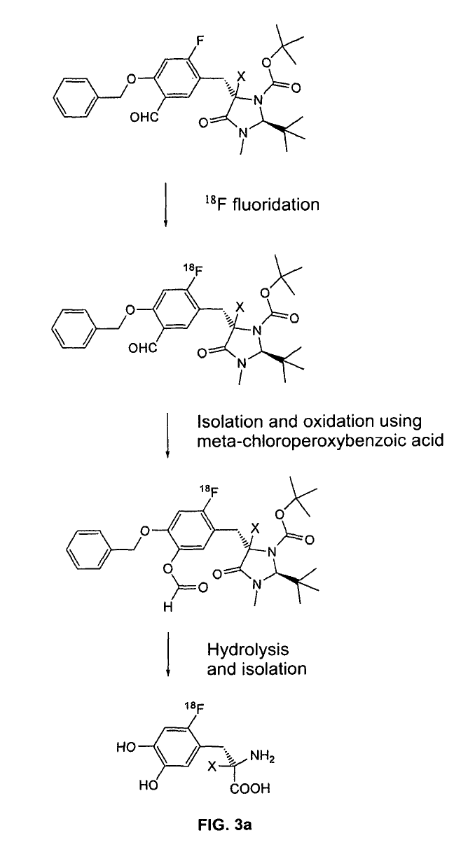

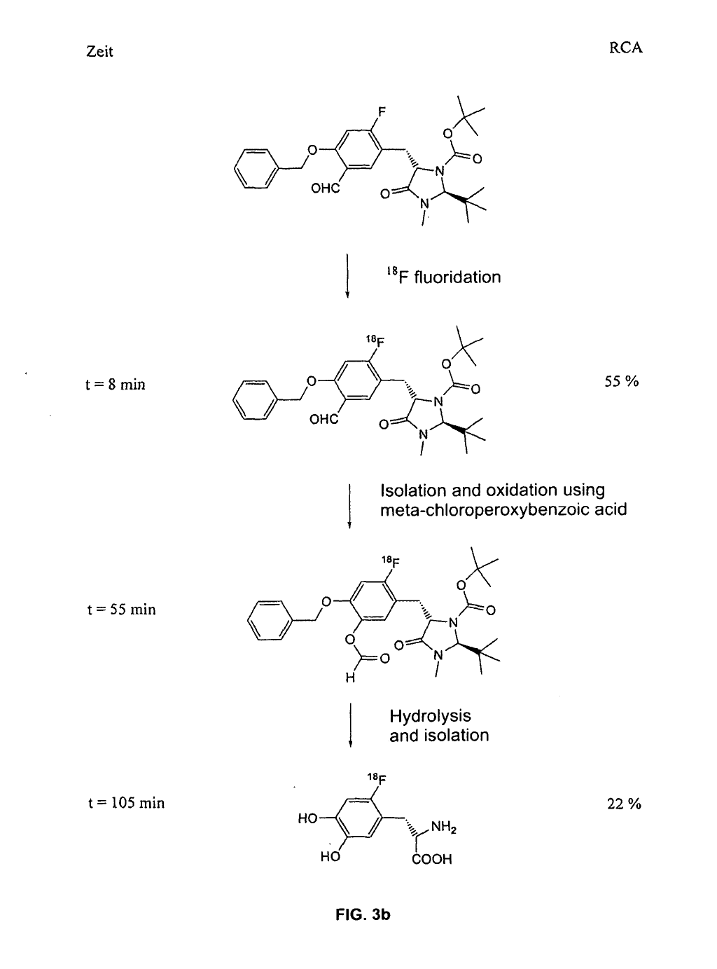

FIG. 3: General steps for producing [¹⁸F]FDOPA and the α-methylated derivative.

FIG. 3a: Exemplary production of [¹⁸F]FDOPA and the α-methylated derivative.

FIG. 3b: Steps for producing [¹⁸F]FDOPA (example).

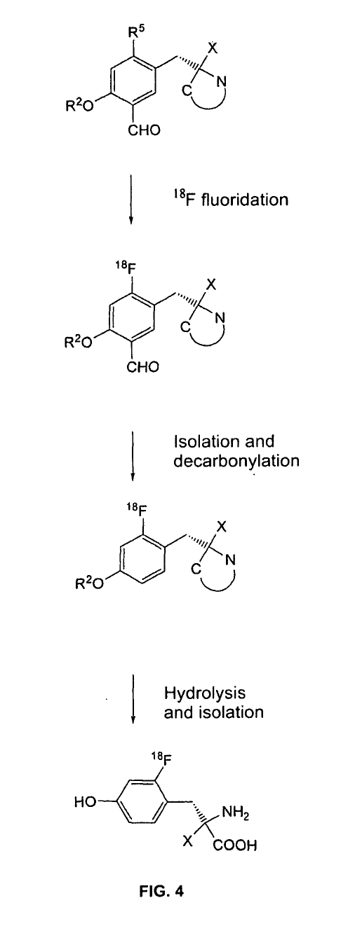

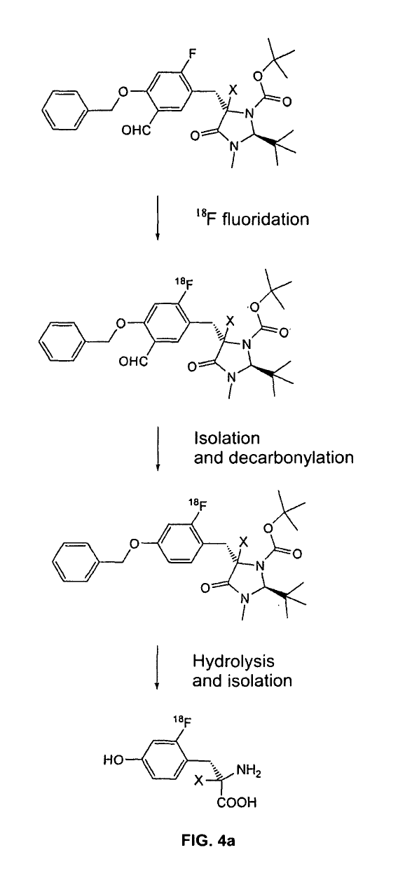

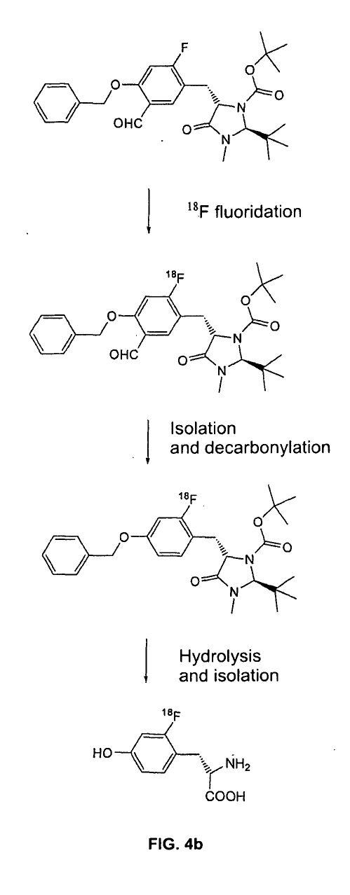

FIG. 4: General steps for producing [¹⁸F]FTyr and the α-methylated derivative.

FIG. 4a: Exemplary production of [¹⁸F]FTyr and the α-methylated derivative.

FIG. 4b: Steps for producing L-[¹⁸F]FTyr (example).

Formula 1 shows the structure of [¹⁸F]FDOPA and the α-methylated derivative. In the formula, X═H or CH₃.

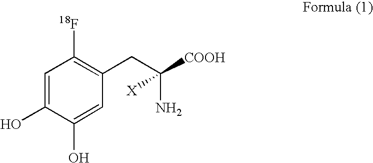

Formula 2 shows the structure of [¹⁸F]FTyr and the α-methylated derivative. In the formula, X═H or CH₃.

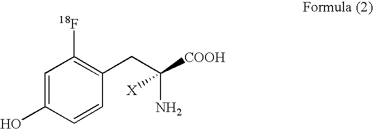

Formula (3) shows the structure of the precursor. In the formula, X═H or CH₃.

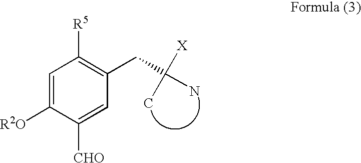

The invention will be explained hereinafter in the general terms.

In the formulas and in the figures, the following groups may be provided as the substituents Rⁿ, X:

R¹=Br, I

R²=benzyl (Bn), methyl (Me)

R³=tetrahydropyranyl (THP), methylthiomethyl (MTM), methoxymethyl (MOM), TBDMS, TBDPS, general silyl protecting groups

R⁴=(S)-BOC-BMI: (S)-1-(tert-butoxycarbonyl)-2-tert-butyl-3-methyl-4-imidazolidinone,

(S)-Cbz-BMI: (S)-1-(benzoylcarbonyl)-2-tert-butyl-3-methyl-4-imidazolidinone,

(S)-BDI: (S)-tert-butyl 2-tert-butyl-4-methoxy-2,5-dihydroimidazol-1-carboxylate, methyl-(S)-BOC-BMI: (2S,5R)-tert-butyl-2-tert-butyl-3,5-dimethyl-4-oxoimidazolidine-1-carboxylate,

methyl-(S)-Cbz-BMI: (S)-1-(benzoylcarbonyl)-2-tert-butyl-3,5-dimethyl-4-imidazolidinone or

methyl-(S)-BDI: (S)-tert-butyl-2-tert-butyl-5-methyl-4-methoxy-2-hydroimidazol-1-carboxylate

Formula notation:

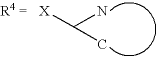

R⁵=nucleophilic leaving group, for example, F, Br, Cl, NO₂, —NR₃⁺, where R=alkyl, such as

CH₃, C₂H₅

X═H or CH₃

FIG. 1 illustrates the general chemical equation according to which the precursor for [¹⁸F]FDOPA and [¹⁸F]FTyr according to formula 3 can be produced.

In preparation for the synthesis of the precursor according to the invention, a compound according to formula a is halogenated into product b in accordance with step i.

In particular bromine or iodine can be used as halogens.

The halogenation can be carried out at a temperature range between −20° C. and −80° C., and preferably between −50° C. and −80° C., with −78° C. being particularly preferred, this being the dry ice temperature.

The halogenation is preferably carried out by adding a buffer.

Sodium acetate may be used as a buffer, for example.

Methanol, ethanol or acetic acid can be used as solvents without buffer.

In a further step ii, the compound of formula b is protected at the carboxylic acid group, and preferably esterified. Esterification can preferably be carried out using a methyl group. For this purpose, trimethylsilyl diazomethane can be used as the methylation reagent, for example.

The solvent used can be at least one component from the group consisting of methanol or chloroform.

A mixture of methanol and chloroform is preferred. A mixing ratio of 1:5 is particularly preferred.

The reaction can be carried out at room temperature.

As an alternative, esterification into an ethyl ester may performed.

The OH group of the ester of the compound according to formula c is provided with a protecting group in step iii.

Benzyl groups or methyl groups may be used as protecting groups.

The introduction of a methyl group as a protecting group may be carried out by a conversion using methyl iodide or methyl bromide.

A benzyl group may be introduced using benzyl bromide or benzyl iodide. The solvent can, in principle, be freely selected. However, it is possible, for example, to use acetone or halogenated hydrocarbon, such as chloromethane, methylene chloride, or chloroform.

The reaction is preferably carried out under reflux.

The conversion is preferably carried out in the presence of a base such as potassium carbonate or amines, such as primary, secondary or tertiary amines or NaH.

The solvent can be freely selected. For example, acetone or halogenated hydrocarbon, such as chloromethane, methylene chloride, or chloroform may be used.

The conversion is preferably carried out in the presence of a base, such as potassium carbonate.

The production method can be used, by way of example, to produce the compound d as a starting material for the production method for the precursor according to the invention. However, it is also possible to pursue different synthesis paths for d.

The ester group of compound d is reduced according to the invention.

The reduction can be carried out with a hydride, for example, and lithium aluminum hydride is particularly preferred.

The hydration is preferably carried out at room temperature.

The solvent used can be an ether, such as diethyl ether or THF. Preferably, diethyl ether is used as the solvent.

The resulting alcohol e is protected in step v with a protecting group.

THP, MTM, or MOM may be used as protecting groups.

In addition, p-toluene sulfonic acid can be added as a catalyst.

If R² is a methyl group, R³ may be tert-butyldimethylsilyl (TBDMS).

The solvent used can be dichloromethane or tetrahydrofurane.

The reaction temperature preferably ranges between 0° C. and room temperature, for example 17° C. to 25° C.

In a subsequent step vi, the substituent R¹ of compound f is replaced by a formyl group.

The formylation can be carried, for example, using anilide, formyl piperidine or dimethylformamide in the presence of metallizing reagents, such as sec-butyl lithium, n-butyl lithium, tert-butyl lithium, lithium or magnesium.

The solvent used can be tetrahydrofurane or another ether, for example.

The reaction can be carried out in a temperature range between −20° C. and −80° C., preferably between −50° C. and −80° C., with −78° C. being particularly preferred, this being the dry ice temperature.

The resulting compound g is reduced to an alcohol h in a subsequent step vii.

The reducing agent used can be a metal hydride, such as sodium borohydride or lithium aluminum hydride, for example.

Suitable solvents are methanol or other alcohols, in particular when using sodium borohydride.

The reaction is preferably carried out at room temperature.

The alcohol h is halogenated or tosylated into compound i in the subsequent reaction viii.

For this purpose, preferably tetrabromomethane is used in the presence of triphenylphosphine as an oxygen scavenger.

The solvent used can be dichloromethane or, halogenated hydrocarbons in general.

The preferred temperature is between 0° C. and approximately 4° C.

In reaction step ix, compound i is converted using a chiral amino acid reagent. To this end, compound i is converted using (S)-BOC-BMI: (S)-1-(tert-butoxycarbonyl)-2-tert-butyl-3-methyl-4-imidazolidinone, (S)-Cbz-BMI: (S)-1-(benzoylcarbonyl)-2-tert-butyl-3-methyl-4-imidazolidinone, (S)-BDI: (S)-tert-butyl 2-tert-butyl-4-methoxy-2,5-dihydroimidazol-1-carboxylate, methyl-(S)-BOC-BMI: (2S,5R)-tert-butyl-2-tert-butyl-3,5-dimethyl-4-oxoimidazolidine-1-carboxylate, methyl-(S)-Cbz-BMI: (S)-1-(benzoylcarbonyl)-2-tert-butyl-3,5-dimethyl-4-imidazolidinone, or methyl-(S)-BDI: (S)-tert-butyl-2-tert-butyl-5-methyl-4-methoxy-2-hydroimidazol-1-carboxylate. The conversion can be carried out in the presence of lithium diisopropylamine.

The solvent used can be tetrahydrofurane or ether, preferably diethylether, or at least one constituent of this class.

The resulting compound j is deprotected in step x at the OR³ function.

For this purpose, pyridinium-p-toluene sulfonic acid can be used, for example. However, it is possible to employ any known method for removing the protecting group, such as the use of acids or MgBr₂.

Possible solvents are alcohols, such as ethanol.

The reaction product k is oxidized into an aldehyde.

For this purpose, known mild oxidation methods may be employed.

The reaction takes place in a range of −20° C. to −80° C., −30° C. to −80° C., or preferably between −50° C. and −80° C. Typically it is conducted at the dry ice temperature of approximately −78° C.

For this purpose, by way of example, Swern oxidation can be carried out. The conversion is carried out using oxalyl chloride or dimethyl sulfoxide in the presence of triethylamine.

The solvent used can be halogenated hydrocarbon, such as dichloromethane.

The reaction product is the precursor of formula 3 according to the invention.

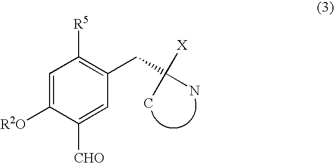

In a further conversion, the precursor having formula 3 can be converted into [¹⁸F]FDOPA or [¹⁸F]FTyr.

To this end, the fluorine atom or another nucleophilic leaving group of the precursor according to formula 3 is substituted by ¹⁸F.

This fluoridation can be attained using standard methods. To this end, the phase transfer catalysts Kryptofix potassium oxalate or tetrabutyl ammonium hydrogen carbonate can be employed as the anion activator for [¹⁸F]fluoride.

The ¹⁸F-fluoridated intermediate product is separated in a further step, and in the case of the [¹⁸F]FDOPA and the α-methylated derivative, is oxidized into ester.

The separation can be carried by way of solid phase extraction. For this purpose, the reaction mixture is purified using a reverse-phase cartridge.

The oxidation of the aldehyde group can be carried out using meta-chloroperoxybenzoic acid or peracetic acid or perborate. The solvents used can be halogenated hydrocarbons, such as chloroform or methylene chloride.

In the case of [¹⁸F]FTyr or the α-methylated derivative thereof, a decarbonylation step is carried out in place of oxidation.

Advantageously, suitable catalysts for the decarbonylation notably comprise one or more transition metals of the secondary groups I, II, VI, VII, and VIII, such as chromium, manganese, nickel, copper or zinc, and preferably one or more metals from the group consisting of the platinum metals, in particular rhodium. In a heterogeneous system, solid catalysts on carriers may be used, or in homogeneous systems, this can be carried out in the liquid phase.

Soluble rhodium complexes, which can be used in a homogeneous liquid system or by which carriers can be impregnated, are, for example, rhodium(I) complexes such as CIRh(PPh₃)₃ (“Wilkinson catalyst”), CIRh(CO) (PPh₃)₂, [CIRh(CO)₂]₂, acacRh(CO) (PPh₃), acacRh(CO)₂, (C₅H₅)Rh(C₈H₁₄) and (C₃H₅)Rh(PPh₃), where Ph is phenyl, acac is acetylacetonate, C₈H₁₄ cyclooctene, C₅H₅ cyclopentadienyl, and C₃H₅ is allyl. Also suited are rhodium(II) and rhodium(III) complexes, such as rhodium(II)-acetate, rhodium(II)-2,4-difluorobenzoate, Rh(acac)₃, RhCl₃′ xH₂O, Rh(NO₃)₃ and (C₃H₅)RhCl₂(PPh₃)₂. Advantageously, compounds which can act as ligands, such as phosphanes, phosphites or amines, may be added to these rhodium complexes.

In a further step, the ester that is obtained is subjected to hydrolysis, whereby [¹⁸F]FDOPA or [¹⁸F]FTyr or the α-methylated derivative thereof is obtained.

The hydrolysis can be carried out in an aqueous solution, and preferably concentrated HI or HBr, or in a solution with KI or HBr.

The product can be separated using HPLC.

An object of the invention is also the compound of formula 3, where X═H or CH₃ and R⁵=a nucleophilic leaving group, such as F, Br, Cl, NO₂, —NR₃⁺, where R=alkyl, such as CH₃, C₂H₅, R²=benzyl (Bn), methyl (Me), and R⁴=(S)-BOC-BMI: (S)-1-(tert-butoxycarbonyl)-2-tert-butyl-3-methyl-4-imidazolidinone, (S)-Cbz-BMI: (S)-1-benzoylcarbonyl-2-tert-butyl-3-methyl-4-imidazolidinone, (S)-BDI: (S)-tert-butyl 2-tert-butyl-4-methoxy-2,5-dihydroimidazol-1-carboxylate, methyl-(S)-BOC-BMI: (2S,5R)-tert-butyl-2-tert-butyl-3,5-dimethyl-4-oxoimidazolidine-1-carboxylate, methyl-(S)-Cbz-BMI: (S)-1-(benzoylcarbonyl)-2-tert-butyl-3,5-dimethyl-4-imidazolidinone, or methyl-(S)-BDI: (S)-tert-butyl-2-tert-butyl-5-methyl-4-methoxy-2-hydroimidazol-1-carboxylate.

Formula notation:

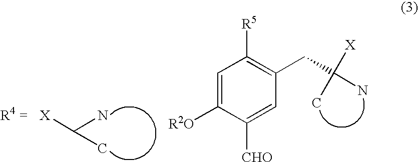

By using the novel labeling precursor, it is possible to produce an enantiopure product having formula 3 (ee≧98%) by way of a nucleophilic synthesis using only three radioactive steps. This allows for automated routine synthesis. The radiochemical yields are 22%. Likewise, the end products, [¹⁸F]FDOPA or [¹⁸F]FTyr, can be obtained in an enantiomeric purity of 98%.

The method according to the invention and the precursor according to formula 3 allow synthesis to be carried out in an automated fashion.

In general, apparatuses for an automated synthesis comprise a charge vessel, which is supplied with reagents from reservoir vessels using a control unit, the reservoir vessels being connected to the charge vessel by way of feed lines. The charge vessel is generally filled and emptied by generating a positive pressure or a negative pressure. By way of example, the commercially available apparatus TRACERlab FX F-N shall be mentioned, in which in addition to reservoir vessels for reagents, a charge vessel made of glassy carbon, a magnetic stirrer, and a retractable needle, is equipped with an activity detector and a vacuum system having a cooling trap. The apparatus comprises an [¹⁸O] water processing unit and a solid phase extraction unit with preparative HPLC, two HPLC eluents and HPLC flow control, UV and radioactivity detectors for the HPLC, collection vessels for fractions, solid phase extraction and HPLC solvent recirculation. Similar apparatuses are known from “One-step high-radiochemical-yield synthesis of [¹⁸F]FP-CIT using a protic solvent System” in Nuc. Med. Biol., 2007; 34: 345-351 by S. Lee, S Oh, D. Chi, S. Kang, H. Kil, J Kim, D. Moon and furthermore from Chen X, Park r, Shahinian A H, et al. ¹⁸F-labeled RGD peptide: initial evaluation for imaging brain tumor angiogenesis/Nuc. Med. Biol. 2004; 31: 179-189.

The precursor according to the invention enables a fully automated implementation of such a system, in which an ¹⁸F-fluoridation of the precursor according to formula 3 is carried out and thereafter the processing into the end product, this being [¹⁸F]FDOPA or [¹⁸F]FTyr, takes place. [¹⁸F]FDOPA and [¹⁸F]FTyr are obtained in an enantiomeric purity of 98%.

### EXAMPLE I

The synthesis according to FIG. 1 can be carried out using the following reagents:

i) Methanol, sodium acetate, bromine

ii) Methanol, chloroform, trimethylsilyl diazomethane

iii) Acetone, potassium carbonate, methyl iodide

iv) Diethylether, lithium aluminum hydride

v) Dichloromethane, dihydropyrane, p-toluene sulfonic acid

vi) Tetrahydropyrane, sec-butyl lithium, dimethylformamide

vii) Methanol, sodium borohydride

viii) Dichloromethane, tetrabromomethane, triphenylphosphine

ix) Tetrahydropyrane, lithium diisopropylamine, (S)-Boc-BMI

x) Ethanol, pyridinium-p-toluene sulfonic acid

xi) Dichloromethane, oxalyl chloride, dimethyl sulfoxide, triethylamine

### SPECIFIC EXEMPLARY EMBODIMENT

5-bromine-4-fluoro-2-hydroxybenzoic acid

1 g (6.4 mmol) of 4-fluoro-2-hydroxybenzoic acid and 2.2 g (26.88 mmol) sodium acetate are dissolved in 10 ml absolute methanol and cooled to −70° C. After adding 0.33 ml (6.4 mmol) bromine in 10 ml methanol in 30 minutes, the reaction solution is heated to room temperature, the solvent is removed under vacuum, and the residue is taken up in diluted hydrochloric acid. The resulting precipitation is suctioned off, washed with water, and taken up in acetic acid ethyl ester. After drying with sodium sulfate, the solvent is removed under vacuum and the pure product is obtained:

Form: colorless solid matter

Yield: 1.23 g (5.2 mmol; 82%)

R_{f}: 0.50 (chloroform/methanol=2:1+0.1% TEA)

Methyl-5-bromine-4-fluoro-2-hydroxybenzoate

1.23 g (5.2 mmol) 5-bromine-4-fluoro-2-hydroxybenzoic acid is dissolved in 10 ml anhydrous dichloromethane and 2 ml absolute methanol, mixed with 3.4 ml (6.76 mmol) trimethylsilyl diazomethane, and stirred for 30 minutes at room temperature. The volatile constituents are removed under vacuum and the product is obtained in pure form.

Form: colorless solid matter

Yield: 1.27 g (5.1 mmol; 99%)

R_{f}: 0.85 (diethyl ether/petroleum ether=1:2).

Methyl-2-(benzyloxy)-5-bromo-4-fluorobenzoate

A suspension of 12.7 g (51 mmol) methyl-5-bromo-4-fluoro-2-hydroxybenzoate, 6.7 ml (56.1 mmol) benzyl bromide and 14.01 g (102 mmol) potassium carbonate in 100 ml acetone is heated for 12 hours under reflux. After cooling, the potassium carbonate is filtered and washed with acetone, the solvent is removed under vacuum, and the raw product is purified by way of column chromatography on silica gel using diethyl ether/petroleum ether 1:3.

Form: colorless solid matter

Yield: 13.14 g (38.76 mmol; 76%)

R_{f}: 0.58 (diethyl ether/petrol ether=1:3).

[2-(benzyloxy)-5-bromo-4-fluorophenyl]methanol

23.25 ml (23.25 mmol) lithium aluminum hydride solution (1M in diethyl ether) is slowly added dropwise under an argon atmosphere to a solution of 13.14 g (38.76 mmol) methyl-2-(benzyloxy)-5-bromo-4-fluorobenzoate in 100 ml anhydrous diethyl ether. Thereafter, this is heated for 60 minutes under reflux, then cooled, and the reaction solution is poured into ice water and acidified until the precipitation dissolves. The reaction mixture is extracted using diethyl ether, washed with sodium chloride and dried over sodium sulfate. The pure product is obtained after removing the solvent.

Form: colorless solid matter

Yield: 10.25 g (32.95 mmol; 85%)

R_{f}: 0.58 (diethyl ether/petroleum ether=1:3).

2[2-(benzyloxy)-5-bromo-4-fluorobenzyloxy]tetrahydro-2H-pyrane

A solution of 10.25 g (32.95 mmol) [2-benzyloxy)-5-bromo-4-fluorophenyl]methanol and 1.9 ml (20.5 mmol) 3,4-dihydro-2H-pyrane in dichloromethane is mixed at 0° C. with a small amount, covering the tip of a spatula, of toluene sulfonic acid monohydrate and stirred for 45 minutes. The reaction solution is mixed with diethyl ether, washed with a sodium chloride solution, a sodium carbonate solution and water, and then again with a sodium chloride solution, then dried over sodium sulfate, and the solvent is removed under vacuum.

Form: colorless oil

Yield: 12.76 g (32.29 mmol; 98%)

R_{f}: 0.75 (diethyl ether/petroleum ether=1:2).

4-(benzyloxy)-2-fluoro-5-[tetrahydro-2H-pyrane-2-yloxy]methyl)benzaldehyde

12.76 g (32.29 mmol) 2-[2-(benzyloxy)-5-bromo-4-fluorobenzyloxy]tetrahydro-2H-pyrane is dissolved in 70 ml absolute tetrahydrofurane, slowly mixed with 24.2 ml (33.9 mmol) sec-BuLi (1.4 M in cyclohexane) at −78° C. under an argon atmosphere, and stirred for 45 minutes. After adding 3.5 ml (45.21 mmol) of dimethyl formamide, the reaction solution is stirred for another 60 minutes at room temperature. After adding water, extraction is performed using diethyl ether, the organic phase is dried over sodium sulfate, and the solvent is removed under vacuum. The raw product is subjected to chromatography on silica gel using diethyl ether/petroleum ether 1:3.

Form: colorless solid matter

Yield: 4.33 g (12.59 mmol; 39%)

R_{f}: 0.53 (diethyl ether/petroleum ether=1:3)

[4-benzyloxy-2-fluoro-5-(tetrahydro-pyrane-2-yloxymethyl)-phenyl]methanol

A solution of 4.33 g (12.59 mmol) 4-(benzyloxy)-2-fluoro-5-[tetrahydro-2H-pyrane-2-yloxy]methyl)benzaldehyde in 50 ml anhydrous methanol is mixed in portions with 0.71 g (18.89 mmol) sodium borohydride while stirring, and stirred for 30 minutes at room temperature. After adding water, extraction is performed using diethyl ether, the organic phase is dried over sodium sulfate, and the solvent is removed under vacuum.

Form: colorless oil

Yield: 4.27 g (12.34 mmol; 98%)

R_{f}: 0.43 (diethyl ether/petroleum ether=1:1)

2-(2-benzyloxy-5-bromomethyl-4-fluoro-benzyloxy-tetrahydro-pyrane 4.53 g (17.27 mmol) triphenylphosphine is slowly added to an ice-cold solution of 4.27 g (12.34 mmol) [4-benzyloxy-2-fluoro-5-(tetrahydro-pyrane-2-yloxymethyl)-phenyl]methanol and 5.11 g (15.42 mmol) tetrabromomethane in 100 ml anhydrous dichloromethane over 10 minutes and stirred for 45 minutes. The reaction solution is mixed with pentane, the precipitate is suctioned off and washed with dichloromethane. The filtrate is washed with 5% sodium hydrogen carbonate solution, water and sodium chloride solution and dried over magnesium sulfate. After removing the solvent, the residue is purified by way of column chromatography on silica gel using diethyl ether/petroleum ether 1:5.

Form: colorless oil

Yield: 1.95 g (4.77 mmol; 39%)

R_{f}: 0.67 (diethyl ether/petroleum ether=1:5)

tert-butyl-5{4-(benzyloxy)-2-fluoro-5-[(tetrahydro-2H-pyrane-2-yloxy)methyl]benzyl-2-tert-butyl-3-methyl-4-oxoimidazolidine-1-carboxylate

3.2 ml (4.77 mmol, 1.5 M in THF) lithium diisopropyl amide is added to a solution of 1.22 g (4.77 mmol) BOC-BMI in anhydrous diethyl ether at −78° C. under an argon atmosphere and stirred for 40 minutes. After adding 1.95 g (4.77 mmol) 2-(2-benzyloxy-5-bromomethyl-4-fluoro-benzyloxy-tetrahydro-pyrane, the reaction solution is stirred for 18 hours at room temperature, mixed with saturated NH₄Cl solution, and taken up in diethyl ether and water. The aqueous phase is extracted two times using diethyl ether; the combined purified organic extracts are dried over NaSO₄, and the solvent is reduced under vacuum. The residue is then subjected to chromatography on silica gel using diethyl ether/petroleum ether 2:1.

Form: colorless foam

Yield: 0.75 g (1.29 mmol; 27%)

R_{f}: 0.69 (diethyl ether/petrol ether=2:1)

tert-butyl-5-[4-benzyloxy-2-fluoro-5-(hydroxymethyl)benzyl]2-tert-butyl-3-methyl-4-oxoimidazolidine-1-carboxylate

0.75 g (1.29 mmol) tert-butyl-5{4-(benzyloxy)-2-fluoro-5-[(tetrahydro-2H-pyrane-2-yloxy)methyl]benzyl-2-tert-butyl-3-methyl-4-oxoimidazolidine-l-carboxylate is dissolved in 30 ml ethanol, mixed with 28 mg (0.13 mmol) pyridinium p-toluenesulfonate and stirred for 18 hours at 55° C. After cooling, the solvent is removed, the residue is taken up in diethyl ether, the organic phase is washed with sodium chloride solution and dried over magnesium sulfate. The solvent is removed under vacuum and the residue is purified by way of column chromatography on silica gel using diethyl ether/petroleum ether 5:1.

Form: colorless foam

Yield: 0.60 g (1.20 mmol; 93%)

R_{f}: 0.78 (diethyl ether/petroleum ether=2:1)

tert-butyl-5-(4-benzyloxy-2-fluoro-formylbenzyl)-2-tert-butyl-3-methyl-4-oxoimidazolidine-1-carboxylate

Under an argon atmosphere, 0.11 ml (1.32 mmol) oxalyl chloride in 1 ml dichloromethane is slowly mixed with 0.2 ml (2.88 mmol) dimethyl sulfoxide at −60° C. and stirred for 10 minutes. After adding 0.60 g (1.20 mmol) tert-butyl-5-[4-benzyloxy-2-fluoro-5-(hydroxymethyl)benzyl]2-tert-butyl-3-methyl-4-oxoimidazolidine-1-carboxylate in 5 ml dichloromethane, it is stirred for another 15 minutes, the reaction solution is mixed with 0.83 ml (6 mmol) triethylamine, slowly heated to room temperature, and after adding 5 ml water it is stirred for another 10 minutes. The aqueous phase is separated and extracted with dichloromethane. The organic phase is dried over sodium sulfate, the solvent is removed under vacuum, and the residue is chromatographed on silica gel using diethyl ether/petroleum ether 2:1.

Form: colorless foam

Yield: 0.42 g (0.85 mmol; 71%)

R_{f}: 0.78 (diethyl ether/petroleum ether=2:1)

Active Synthesis

In a conical reaction vessel (Rectivial, 5 ml) having magnetic stirrer rods, 130 μl tetrabutyl ammonium hydrogen carbonate is added to 0.9 ml absolute acetonitrile (for DNA synthesis, Merck) to dry the aqueous [¹⁸F]fluoride solution. The reactor is closed with a screw cap having a silicone septum, through which two single-use cannulas are pierced for the vacuum and argon connections. The solution is then evaporated at reduced pressure at a temperature of 80° C. for drying. This azeotropic drying process is repeated two times, each time with 0.8 ml acetonitrile, and then the apparatus is evacuated for 5 minutes. After gasifying the reactor with argon, a temperature of 120° C. is adjusted, 23 μmol tert-butyl-5-(4-benzyloxy-2-fluoro-formylbenzyl)-2-tert-butyl-3-methyl-4-oxoimidazolidine-1-carboxylate is taken up in 0.8 ml N,N-dimethylformamide and transferred into the reaction vessel using a tuberculin syringe. The reaction mixture of the ¹⁸F labeling is taken up in 9 ml water after a reaction time of 8 minutes to separate the solvent and the phase transfer catalyst and is conducted over an LiChrolut® RP-18e cartridge (500 mg). After rinsing the cartridge with 5 ml water, it is dried with a strong argon flow for 2 minutes. The fixed organic constituents are eluted with 1.5 ml acetonitrile in a reactor and dried azeotropically. After adding 22 mg (92 μmol, 77%) mCPBA in 1 ml chloroform, this is stirred for 20 minutes at 60° C. and then the solvent is removed. The residue is mixed with 1 ml 48% HBr and heated for 30 minutes to 150° C. After cooling, the HBr phase is taken up in 1 ml water and the product is purified by way of semi-preparative HPLC.

1. A compound of the formula (3) 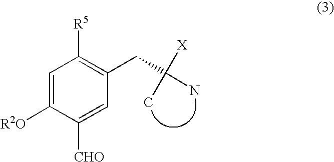
where X═H or CH₃, and R⁵=nucleophilic leaving group, such as F, Br, Cl, NO₂, —NR₃⁺, where R=alkyl, such as CH₃, C₂H₅, R²=benzyl (Bn) or methyl (Me), and R⁴=(S)-BOC-BMI: (S)-1-(tert-butoxycarbonyl)-2-tert-butyl-3-methyl-4-imidazolidinone, (S)-Cbz-BMI: (S)-1-(benzoylcarbonyl)-2-tert-butyl-3-methyl-4-imidazolidinone, (S)-BDI: (S)-tert-butyl 2-tert-butyl-4-methoxy-2,5-dihydroimidazol-1-carboxylate, methyl-(S)-BOC-BMI: (2S,5R)-tert-butyl-2-tert-butyl-3,5-dimethyl-4-oxoimidazolidine-1-carboxylate, methyl-(S)-Cbz-BMI: (S)-1-(benzoylcarbonyl)-2-tert-butyl-3,5-dimethyl-4-imidazolidinone, or methyl-(S)-BDI: (S)-tert-butyl-2-tert-butyl-5-methyl-4-methoxy-2-hydroimidazol-1-carboxylate.

2. A method for producing the compound according to formula (3) 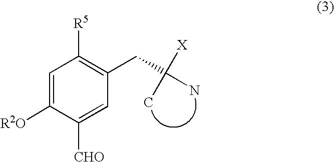
where X═H or CH₃, and R⁵=nucleophilic leaving group, such as F, Br, Cl, NO₂, —NR₃⁺, where R=alkyl, such as CH₃, C₂H₅, R²=benzyl (Bn) or methyl (Me), and R⁴═(S)-BOC-BMI: (S)-1-(tert-butoxycarbonyl)-2-tert-butyl-3-methyl-4-imidazolidinone, (S)-Cbz-BMI: (S)-1-(benzoylcarbonyl)-2-tert-butyl-3-methyl-4-imidazolidinone, (S)-BDI: (S)-tert-butyl 2-tert.-butyl-4-methoxy-2,5-dihydroimidazol-1-carboxylate, methyl-(S)-BOC-BMI: (2S,5R)-tert-butyl-2-tert-butyl-3,5-dimethyl-4-oxoimidazolidine-1-carboxylate, methyl-(S)-Cbz-BMI: (S)-1-(benzoylcarbonyl)-2-tert-butyl-3,5-dimethyl-4-imidazolidinone, or methyl-(S)-BDI: (S)-tert-butyl-2-tert-butyl-5-methyl-4-methoxy-2-hydroimidazol-1-carboxylate,
wherein in a step (iv) a compound (d) 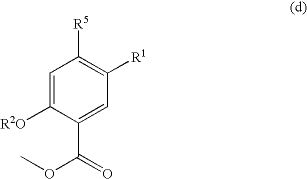
where R⁵=nucleophilic leaving group, such as F, Br, Cl, NO₂, —NR₃⁺, where R=alkyl, such as
CH₃, C₂H₅
R¹=Br or I
and R²=benzyl (Bn) or methyl (Me),
is reduced into compound (e), 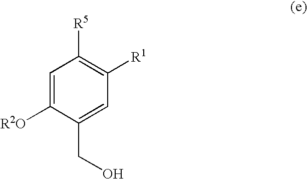
compound (e) in a further step (v) is converted into compound (f) 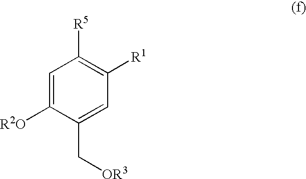
where R³=protecting group=tetrahydropyranyl (THP),
methylthiomethyl (MTM), or methoxymethyl (MOM), TBDMS, TBDPS, silyl protecting groups,
in a step (vi) the group R¹ in compound (f) is substituted by a formyl group, whereby the product (g) is produced, 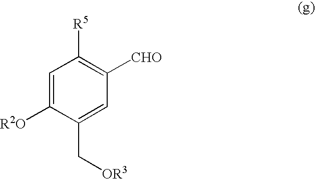
the compound according to formula (g) is reduced to an alcohol (h) in a further step (vii), 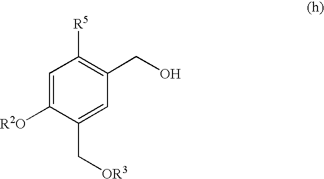
in a further reaction (viii) the alcohol being converted into product (i), 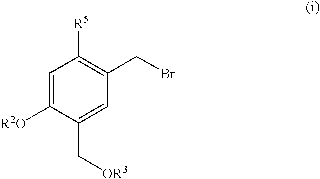
by way of bromation, wherein, as an alternative, instead of the Br group a tosyl group can be introduced by way of tosylation,
the compound (i) in a further step (ix) is converted into compound (j) using a chiral reagent R⁴ from the group consisting of (S)-BOC-BMI: (S)-1-(tert-butoxycarbonyl)-2-tert-butyl-3-methyl-4-imidazolidinone, (S)-Cbz-BMI: (S)-1-(benzoylcarbonyl)-2-tert-butyl-3-methyl-4-imidazolidinone, (S)-BDI: (S)-tert-butyl 2-tert-butyl-4-methoxy-2,5-dihydroimidazol-1-carboxylate, methyl-(S)-BOC-BMI: (2S,5R)-tert-butyl-2-tert-butyl-3,5-dimethyl-4-oxoimidazolidine-1-carboxylate, methyl-(S)-Cbz-BMI: (S)-1-(benzoylcarbonyl)-2-tert-butyl-3,5-dimethyl-4-imidazolidinone, or methyl-(S)-BDI: (S)-tert-butyl-2-tert-butyl-5-methyl-4-methoxy-2-hydroimidazol-1-carboxylate, 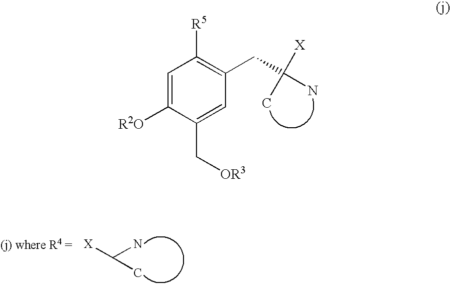
is the formula notation,
which in step (x) is deprotected at the alcohol function (k) 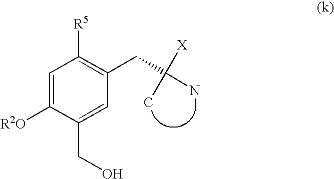
whereupon in step (xi) the alcohol function is converted into the compound according to formula (3) by way of oxidation.

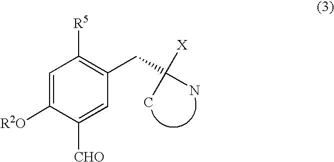

3. A method for producing a compound according to formula (1) and (2), 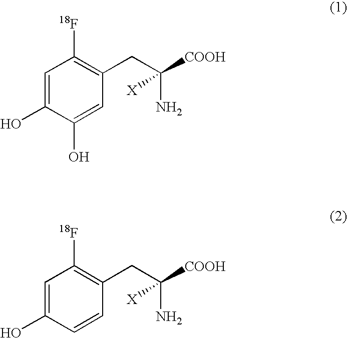
where X═H or CH₃
wherein
the compound according to formula (3) is ¹⁸F fluoridated, separated in a further step, and is oxidized if the compound according to formula (1) is produced, and is decarbonylated if the compound according to formula (2) is produced, and the resulting product is hydrolyzed and separated.

4. The method according to claim 3, wherein the ¹⁸F fluoridation is carried out using a phase transfer catalyst as the anion activator.

5. The method according to claim 3 or 4, wherein the separation of the ¹⁸F fluoridation product is carried out by way of solid phase extraction.

6. A method according to claim 3 or 4, wherein the formyl group is oxidized into ester if the compound according to formula (1) is produced.

7. The method according to claim 6, wherein the oxidation is carried out using meta-chloroperoxybenzoic acid or peracetic acid or perborate.

8. A method according to claim 3 or 4, wherein the ¹⁸F fluoridated precursor is decarbonylated by way of a catalyst to produce the compound according to formula (2).

9. A method according to claim 3 or 4, wherein the ester or the decarbonylated product is hydrolyzed.

10. A method according to claim 3 or 4, wherein the product according to formula (1) or (2) is isolated by means of HPLC.

11. A method according to claim 5, wherein the formyl group is oxidized into ester if the compound according to formula (1) is produced.

12. The method according to claim 5, wherein the oxidation is carried out using meta-chloroperoxybenzoic acid or peracetic acid or perborate.

13. A method according to claim 5, wherein the ¹⁸F fluoridated precursor is decarbonylated by way of a catalyst to produce the compound according to formula (2).

14. A method according to claim 5, wherein the ester or the decarbonylated product is hydrolyzed.

15. A method according to claim 6, wherein the ester or the decarbonylated product is hydrolyzed.

16. A method according to claim 7, wherein the ester or the decarbonylated product is hydrolyzed.

17. A method according to claim 8, wherein the ester or the decarbonylated product is hydrolyzed.

18. A method according to claim 5, wherein the product according to formula (1) or (2) is isolated by means of HPLC.

19. A method according to claim 6, wherein the product according to formula (1) or (2) is isolated by means of HPLC.

20. A method according to claim 7, wherein the product according to formula (1) or (2) is isolated by means of HPLC.

21. A method according to claim 8, wherein the product according to formula (1) or (2) is isolated by means of HPLC.
